SEQUENCE LISTING (36 sequences)

SEQ ID NO: 1 - protein, 273 aa, Artificial Sequence
  Precursor domain of TGFBeta
  PEPTIDE (1)..(273)
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQS

SEQ ID NO: 2 - protein, 6 aa, Artificial Sequence
  Proteolytic cleavage site
  PEPTIDE (1)..(6)
PLGLWA

SEQ ID NO: 3 - protein, 5 aa, Artificial Sequence
  Linker peptide
  PEPTIDE (1)..(5)
GGGGS

SEQ ID NO: 4 - protein, 323 aa, Artificial Sequence
  Fusion peptide
  PEPTIDE (1)..(321)
  PEPTIDE (1)..(323)
MPPSGLRLLPLLLPLLWLLVLTPGRPAAGLSTCKTIDMELVKRKRIEAIRGQILSKLRLASPPSQGEVPPGPLPEAVLALYNSTRDRVAGESAEPEPEPEADYYAKEVTRVLMVETHNEIYDKFKQSTHSIYMFFNTSELREAVPEPVLLSRAELRLLRLKLKVEQHVELYQKYSNNSWRYLSNRLLAPSDSPEWLSFDVTGVVRQWLSRGGEIEGFRLSAHCSCDSRDNTLQVDINGFTTGRRGDLATIHGMNRPFLLLMATPLERAQHLQSEFGGGGSPLGLWAGGGGSAAAQERPPLQQPPHRDKKPCKNFFWKTFSSCK

SEQ ID NO: 5 - protein, 8 aa, Artificial Sequence
  Linker region
  PEPTIDE (1)..(8)
GGGGSAAA

SEQ ID NO: 6 - protein, 14 aa, Artificial Sequence
  CST-29
  PEPTIDE (1)..(14)
PCKNFFWKTFSSCK

SEQ ID NO: 7 - protein, 17 aa, Artificial Sequence
  CST 17
  PEPTIDE (1)..(17)
DRMPCRNFFWKTFSSCK

SEQ ID NO: 8 - protein, 14 aa, Artificial Sequence
  Somatostatin 14
  PEPTIDE (1)..(14)
AGCKNFFWKTFTSC

SEQ ID NO: 9 - protein, 14 aa, Artificial Sequence
  CST analogue
  MISC_FEATURE (7)..(7): 3,5-difluorophenylalanine
  MISC_FEATURE (7)..(7): 3,5-difluorofenilalanina
AGCKNFFWKTFTSC

SEQ ID NO: 10 - protein, 14 aa, Artificial Sequence
  CST analogue
  MISC_FEATURE (6)..(6): 3,5-difluorophenylalanine
  MISC_FEATURE (6)..(6): 3,5-difluorofenilalanina
AGCKNXFWKTFTSC

SEQ ID NO: 11 - protein, 14 aa, Artificial Sequence
  CST analogue
  MISC_FEATURE (11)..(11): 3,5-difluorophenylalanine
AGCKNFFWKTXTSC

SEQ ID NO: 12 - protein, 14 aa, Artificial Sequence
  CST analogue
  MISC_FEATURE (6)..(6): 3,5-difluorofenilalanina
  MISC_FEATURE (11)..(11): 3,5-difluorofenilalanina
AGCRNXFWKTXSSC

SEQ ID NO: 13 - protein, 14 aa, Artificial Sequence
  CST analogue
  MISC_FEATURE (5)..(5): 3,4,5-trimetilfenilalanina
PCKNXFWKTFTSCK

SEQ ID NO: 14 - protein, 14 aa, Artificial Sequence
  CST analogue
  MISC_FEATURE (6)..(6): 3,4,5-trimetilfenilalanina
PCKNFXWKTFTSCK

SEQ ID NO: 15 - protein, 14 aa, Artificial Sequence
  CST analogue
  MISC_FEATURE (6)..(6): 3,5-difluorofenilalanina
PCKNFXWKTFTSCK

SEQ ID NO: 16 - protein, 14 aa, Artificial Sequence
  CST analogue
  MISC_FEATURE (10)..(10): 3,4,5-trimetilfenilalanina
PCKNFFWKTXTSCK

SEQ ID NO: 17 - protein, 14 aa, Artificial Sequence
  CST analogue
  MISC_FEATURE (5)..(5): 3,4,5-trimetilfenilalanina
  MISC_FEATURE (10)..(10): 3,4,5-trimetilfenilalanina
PCRNXFWKTXTSCK

SEQ ID NO: 18 - protein, 14 aa, Artificial Sequence
  CST analogue
  MISC_FEATURE (5)..(5): 3,5-difluorofenilalanina
  MISC_FEATURE (10)..(10): 3,4,5-trimetilfenilalanina
PCKNXFWKTXSSCK

SEQ ID NO: 19 - protein, 13 aa, Artificial Sequence
  CST analogue
  MISC_FEATURE (5)..(5): 3,4,5-trimetilfenilalanina
PCKNXFWKTFTSC

SEQ ID NO: 20 - protein, 13 aa, Artificial Sequence
  CST analogue
  MISC_FEATURE (10)..(10): 3,5-difluorofenilalanina
PCKNFFWKTXTSC

SEQ ID NO: 21 - protein, 15 aa, Artificial Sequence
  CST analogue
  MISC_FEATURE (6)..(6): 3,4,5-trimetilfenilalanina
MPCRNXFWKTFSSCK

SEQ ID NO: 22 - protein, 17 aa, Artificial Sequence
  CST analogue
  MISC_FEATURE (8)..(8): 3,4,5-trimetilfenilalanina
DRMPCRNXFWKTFTSCK

SEQ ID NO: 23 - protein, 17 aa, Artificial Sequence
  CST analogue
  MISC_FEATURE (8)..(8): 3,5-difluorofenilalanina
NRMPCRNXFWKTFTSCK

SEQ ID NO: 24 - protein, 14 aa, Artificial Sequence
  CST analogue
  MISC_FEATURE (6)..(6): 3,4,5-trimetilfenilalanina
PCKNFXWKTFTSCK

SEQ ID NO: 25 - protein, 14 aa, Artificial Sequence
  CST analogue
  MISC_FEATURE (6)..(6): 3,4,5-trimetilfenilalanina
PCKNFXWKTFTSCK

SEQ ID NO: 26 - protein, 14 aa, Artificial Sequence
  CST analogue
  MISC_FEATURE (6)..(6): 3,5-difluorofenilalanina
PCKNFXWKTFTSCK

SEQ ID NO: 27 - protein, 14 aa, Artificial Sequence
  CST analogue
  MISC_FEATURE (10)..(10): 3,4,5-trimetilfenilalanina
PCKNFFWKTXTSCK

SEQ ID NO: 28 - protein, 14 aa, Artificial Sequence
  CST analogue
  MISC_FEATURE (5)..(5): 3,4,5-trimetilfenilalanina
  misc_feature (10)..(10): Xaa can be any naturally occurring amino acid
PCRNXFWKTXTSCK

SEQ ID NO: 29 - protein, 14 aa, Artificial Sequence
  CST analogue
  MISC_FEATURE (5)..(5): 3,5-difluorofenilalanina
  misc_feature (10)..(10): Xaa can be any naturally occurring amino acid
PCKNXFWKTXSSCK

SEQ ID NO: 30 - protein, 13 aa, Artificial Sequence
  CST analogue
  MISC_FEATURE (5)..(5): 3,4,5-trimetilfenilalanina
PCKNXFWKTFTSC

SEQ ID NO: 31 - protein, 13 aa, Artificial Sequence
  CST analogue
  MISC_FEATURE (5)..(5): 3,4,5-trimetilfenilalanina
PCKNXFWKTFTSC

SEQ ID NO: 32 - protein, 13 aa, Artificial Sequence
  CST analogue
  MISC_FEATURE (10)..(10): 3,5-difluorofenilalanina
PCKNFFWKTXTSC

SEQ ID NO: 33 - protein, 15 aa, Artificial Sequence
  CST analogue
  MISC_FEATURE (6)..(6): 3,4,5-trimetilfenilalanina
MPCRNXFWKTFSSCK

SEQ ID NO: 34 - protein, 17 aa, Artificial Sequence
  CST analogue
  MISC_FEATURE (8)..(8): 3,4,5-trimetilfenilalanina
DRMPCRNXFWKTFTSCK

SEQ ID NO: 35 - protein, 17 aa, Artificial Sequence
  CST analogue
  MISC_FEATURE (8)..(8): 3,4,5-trimetilfenilalanina
DRMPCRNXFWKTFTSCK

SEQ ID NO: 36 - protein, 17 aa, Artificial Sequence
  CST analogue
  MISC_FEATURE (8)..(8): 3,4,5-trimetilfenilalanina
DRMPCRNXFWKTFTSCK

(19) United States
(12) Patent Application Publication
(10) Pub. No.: US 2022/0135640 A1
(43) Pub. Date: May 5, 2022

(54) CORTISTATIN OR AN ANALOGUE THEREOF AS A PHARMACEUTICALLY ACTIVE AGENT IN LATENT FORM

(71) Applicant: CONSEJO SUPERIOR DE INVESTIGACIONES CIENTIFICAS (CSIC), Sevilla (ES)

(72) Inventors: Mario DELGADO MORA, Armilla (Granada) (ES); Jenny CAMPOS SALINAS, Armilla (Granada) (ES)

(21) Appl. No.: 17/431,082

(22) PCT Filed: Feb. 17, 2020
(86) PCT No.: PCT/EP2020/054118

§ 371 Date: Aug. 13, 2021

(30) Foreign Application Priority Data
Feb. 15, 2019 (ES) P201930121

(51) Int. Cl. C07K 14/655; A61P 17/00; A61P 11/00; A61P 1/16

(52) U.S. Cl. CPC C07K 14/655; A61P 17/00; A61K 38/00; A61P 1/16; A61P 11/00

(57) ABSTRACT

The blood half-life of endogenous peptides such as somatostatin and cortistatin is extremely short, barely reaching a few In minutes [Skamene et al., Clin. Endocrinol. 1984, 20, 555-564]. Thus, there is a need to find new systems or compositions that comprise cortistatinor an analogue thereof for the treatment of those pathologies in which specific cortistatin receptors and those receptors shared with other molecules like somatostatin (sstr1, sstr2, sstr3, sstr4 and/or sstr5) and/or ghrelin (GHSR) are expressed, being, furthermore, more stable in blood than cortistatin. The present invention providesan improved means for providing cortistatinor an analogue thereof as a pharmaceutically active agent in latent form, more stable in blood than cortistatin that liberates cortistatin in a controlled-release manner.

### TECHNICAL FIELD OF THE INVENTION

The present provides a fusion protein comprising a latency associated peptide (LAP) and cortistatin or an analogue thereof as a pharmaceutically active agent in which the LAP and the pharmaceutically active agent are connected by an amino acid sequence comprising a proteolytic cleavage site.

### BACKGROUND OF THE INVENTION

The present invention relates to the use of proteins, protein derivatives and DNA constructs that confer latency to cortistatin or an analogue thereof as a pharmaceutically active agent where the pharmaceutically agent is released by the action of a MMP. Such products are useful in the treatment of chronic fibrosis, preferably a chronic fibrosis selected from the list consisting of liver fibrosis, dermal fibrosis, lung fibrosis, and Scleroderma.

Most cytokines and growth factors are expressed under tight control mechanisms. Their gene expression is regulated by environmental stimuli such as infection, cell-cell interactions, change in extracellular matrix composition and interactions with adhesion molecules or via stimulation with other cytokines.

In addition to the control at the transcriptional and post-transcriptional level, some is cytokines are not released into the medium unless a second signal activates the cell. A third level of regulation for cytokine activity is found in molecules which are secreted in a latent form and become “activated” by releasing the cytokine moiety where processes of inflammation, wound healing and tissue repair takes place (Khalil N, Microbes and Infection, 1, 1255-1263 (1999). In this latter respect, transforming growth factor beta (TGFβ) has received greatest attention.

TGFβ is synthesized as a dimeric latent cytokine composed of an amino terminal latency associated protein (LAP) and the active TGFβ cytokine at its COOH terminal end (Roberts and Sporn, Peptide Growth Factors and their Receptors: Sporn, M B and Roberts, A B, Springer-Verlag, 419-472 (1996); Roth-Eicchorn et al., Hepatology, 28 1588-1596 (1998)). The precursor peptide contains a signal peptide (residues 1-29) necessary for protein secretion and guiding the molecule through the Golgi apparatus to become processed by proteolytic cleavage and glycosylation. The LAP domain is separated from TGFβ by proteolytic cleavage at arginines (277-278).

Mature TGFβ begins at alanine 279. The LAP, in addition to protect TGFβ, contains important residues necessary for the interaction with other molecules. Mutations in the LAP domain have recently been associated with the autosomal dominant Camurati-Engelmann disease (Janssens et al., Nature Genetics, 26, 273-275 (2000). Cysteines 224 and 226 are important in the intermolecular disulphide bond between two LAPs. Their mutation to serine renders the molecule “active” (Sanderson et al., Proc. Natl. Acad. Sci. USA, 92, 2572-2576 (1995); Brunner et al., Mol. Endocrinol. 6, 1691-1700 (1992); Brunner et al., J. Biol. Chem., 264, 13660-13664 (1989)). The RGD motif (245-247) facilitates the interaction with integrins (Munger et al., Mol, Biol. of the Cell, 9, 2627-2638 (1998; Derynck R, TIBS, 19, 548-553 (1994)). Nucleic acid encoding TGFβ is described in U.S. Pat. No. 5,801,231.

In most cell types studied, including those of mesenchymal, epithelial and endothelial origin, TGFβ is secreted in a latent form consisting of TGFβ and its latency associated peptide (LAP) propeptide dimers, covalently linked to latent TGFβ-binding proteins (LTBPs). LTBPs are also needed for the secretion and folding of TGFβ (Miyazano et al., EMBO J. 10, 1091-1101 (1991); Miyazano et al., J. Biol. Chem. 267, 5668-5675 (1992); Eklov et al., Cancer Res. 53, 3193-3197 (1993)). Cysteine 33 is important for is the disulphide bridge with the third 8 cysteine-rich repeat of latent TGFβ binding protein (LTBP) (Saharinen et al., The EMBO Journal, 15, 245-253 (1996). Modification of LTBP by enzymes such as thrombospondin (Schultz et al., The Journal of Biological Chemistry, 269, 26783-26788 (1994); Crawford et al., Cell, 93, 1159-1170 (1998)), transglutaminase (Nunes et al., J. Cell, Biol. 136, 1151-1163 (1997); Kojima et al., The Journal of Cell Biology, 121, 439-448 (1993)) and MMP9, MMP2 (Yu and Stamenkovic, Genes and Dev, 14, 163-176 (2000)) could release the active portion of TGFβ from the latent complex.

Cytokines are natural products serving as soluble local mediators of cell-cell interactions. They have a variety of pleiotropic actions, some of which can be harnessed for therapeutic purposes. Targeting of cytokines to specific cell types using scFv (Lode et al., Pharmacol. Ther, 80, 277-292 (1998)) and vWF (Gordon et al., Human Gene Therapy, 8, 1385-1394 (1997)) have focused entirely on the active cytokine moiety of the cytokine complex.

Pharmacologically active proteins or other medicines based on such agents, which have to be administered at very high concentrations systemically in order to achieve biologically effective concentrations in the tissue being targeted, tend to give rise to undesirable systemic effects, for example toxicity, which limit their use and efficacy.

The principles underlying the construction of such a system for providing latency to pharmaceutically active agents using the LAP of TGFβ was described in WO 02/055098. The present inventors have now developed an improved means for providing cortistatin or an analogue thereof as a pharmaceutically active agent in latent form based on this system. This is particularly important in the case of cortistatin for the following reasons.

Cortistatin (CST) is a natural endogenous peptide of 14 amino acids, discovered in rats in 1996 [de Lecea et al., Nature, 1996, 381, 242-245] and later in 1997, found in humans as an extended form of 17 amino acids (CST-17) [Fukusimi et al., Biochem. Biophys. Res. Commun, 1997, 232, 157-163]. Cortistatin, in fact, exists in two biologically active forms as its precursor (prepro-CST) gives rise to CST-14 and CST-29 in rodents and to CST-17 and CST-29 in humans.

Cortistatin has a high homology to another endogenous peptide, somatostatin (SST), which is highly conserved and found in mammals in the form of somatostatin-14 (SST-14) and somatostatin-28 (SST-28):

Example sequences of cortistatin and somatostatin:

Cortistatin-29 (rat/mouse)
(SEQ ID NO 6)
H2N-Pc[CKNFFWKTFSSC]K-OH
Cortistatin-17 (human)
(SEQ ID NO 7)
H2N-DRMPc[CRNFFWKTFSSC]K-OH
Somatostatin-14 (human/rat/mouse)
(SEQ ID NO 8)
H2N-AGc[CKNFFWKTFTSC]-OH

In fact, cortistatin interacts with the 5 G protein-coupled membrane receptors described for somatostatin, sstr1-sstr5 [a) Spier et al., Brain Research Reviews 2000, 33, 228-241; b) Patel et al., Endocrinology 1994, 135, 2814-2817]. But cortistatin is not somatostatin [Gonzalez-Rey et al., Mol. Cell. Endocrinol. 2008, 286 (1-2), 135-140], and thus, in addition to its nanomolar affinity to somatostatin receptors, cortistatin also interacts with the Ghrelin receptor (GHSR).

Furthermore, in the search for a specific receptor for cortistatin, the orphan receptor MrgX2 was described as the first human specific receptor for cortistatin [Robas et al., J. Biol. Chem. 2003, 278, 44400-44404]. Subsequently, the absence of this receptor in cells of the immune system and its high affinity for other neuropeptides, such as proadrenomedullin, have made that nowadays it is not considered as a specific cortistatin receptor [van Hagen et al., Mol. Cell. Endocrinol. 2008, 286(1-2), 141-147] and characterisation of a specific cortistatin receptor is an issue that remains unresolved.

Cortistatin's immunomodulatory activity has been widely demonstrated in experimental models of diseases that course with inflammatory and autoimmune responses such as Lethal Endotoxin Shock, Crohn's Disease and Rheumatoid arthritis [a) Gonzalez-Rey et al., J. Exp. Med. 2006, 203(3), 563-571; b) Gonzalez-Rey et al., Proc. Natl. Acad. Sci. USA 2006, 103, 4228-4233; c) Gonzalez-Rey et al., Ann. Rheum. Dis. 2007, 66 (5), 582-588; d) WO 2007/082980 A1]. Said immunoregulatory action may be correlated with its expression in lymphocytes, monocytes, macrophages and dendritic cells and cells of the immune system [a) Dalm V. A. et al., Am. J. Physiol. Endocrinol. Metab. 2003, 285, E344-353; b) Dalm V. A. et al., J. Clin. Endocrinol. Metab. 2003, 88, 270-276]. The expression of cortistatin and its receptors in the human immune system and pathologies of the immune system has recently been reviewed [van Hagen et al., Mol. Cell. Endocrinol. 2008, 286(1-2), 141-147].

In the above referenced research studies that showed cortistatin's efficacy in diseases with inflammatory and immune component, CST-29 was used. CST-29 is a long endogenous peptide, of high synthetic difficulty and therefore low industrial viability for its industrial application in the pharmaceutical sector. Its pharmaceutical use also presents an additional problem: its low serum stability.

Other proposals under study prove the efficacy of the endogenous peptide CST-17 combined with the neuropeptide EI for the treatment of inflammatory and autoimmune diseases [WO 2009/043523 A2], which presents the advantage of a lower synthetic difficulty for its industrial use. However, it still possesses the disadvantage of having a low stability in serum due to its native structure with L-amino acids.

Generally, peptide based drugs are advantageous because peptides are intrinsically non-toxic, their efficacy at low doses ensures that they do not cause significant side effects in comparison to other drugs based on small molecules or on antibodies, but they do have to be modified to improve their bioavailability and half-life. The incorporation of non-natural amino acids into the natural sequence is one of the strategies known in prior art for increasing an endogenous peptide's stability. For example, modifications of somatostatin with halogenated amino acids, with p-chloro-Phe and pentafluoro-Phe in positions 6, 7 and 11, have been described [WO 2007/081792 A2; Meyers C. A. et al., Digestion 1981, 21(1), 21-4]. The same positions 6, 7 and 11 of original somatostatin have also been modified with mesitylalanine and mesitylglycine, resulting in somatostatin analogues that are more stable [WO 2010/128098 A1]. However these stabilizing modifications may compromise the functionality of the original molecule. This is the case of octreotide, a somatostatin analogue in clinical use that is much more stable than the original molecule, which keeps binding to the sstrt2 receptor but completely loses its affinity to the sstr1 and sstr4 receptors. [Patel et al., Endocrinology 1994, 135, 2814-2817].

The blood half-life of endogenous peptides such as somatostatin and cortistatin is extremely short, barely reaching a few minutes [Skamene et al., Clin. Endocrinol. 1984, 20, 555-564]. Thus, there is a need to find new systems or compositions that comprise cortistatin or an analogue thereof for the treatment of those pathologies in which specific cortistatin receptors and those receptors shared with other molecules like somatostatin (sstr1, sstr2, sstr3, sstr4 and/or sstr5) and/or ghrelin (GHSR) are expressed, being, furthermore, more stable in blood than cortistatin. This is the reason why the present inventors have now developed an improved means for providing cortistatin or an analogue thereof as a pharmaceutically active agent in latent form.

### BRIEF DESCRIPTION OF THE FIGURES

The present invention will now be described by way of example only with reference to the accompanying figures wherein:

FIG. 1 is a hypothetical representation of LAP-GS-MMP-GS-CST29r and its putative folding and interaction with LTBP.

FIG. 2 illustrates the experiments performed wherein sclerodermia was induced by intradermal injection of bleomycin (3 times per week, during four weeks) in an area of 1 cm³ in the dorsal skin of C57Bl/6 mice. Mice were locally treated around the lesion area with saline (group bleomycin), with cortistatin (group belomycin+CST, 3 time per week, 10 ng each time), with empty LAP vector (Bleomycin+LAP, once a week, 20 pg) or with LAP-CST (Bleomycin+LAP-CST, once a week, 20 pg). Naïve animals without bleomycin were used as basal control reference. After four weeks, lesioned skin area was dissected and processed for histological analysis using Mason Trichromic staining. Skin thickness (from epidermis to hypodermis) was quantified using Image J program. Fibrotic deposits in skin are stained in blue in sections.

FIG. 3 illustrates the experiments performed wherein lung fibrosis was induced by intratracheal injection of bleomycin (50 μg/kg body weight, dissolved in 50 μl of saline) in C57Bl/6 mice. Mice were treated by nasal inhalation of saline (group bleomycin+saline), cortistatin (group belomycin+CST, 3 times per week, 10 ng each time), or LAP-CST (Bleomycin+LAP-CST, once a week, 20 pg). After 18 days, lungs were dissected and processed for histological analysis using Mason Trichromic staining and Sirius Red staining. Lung fibrosis and tissue damage were quantified using Image J program and scored using an established clinical index from 0 to 4 in a blinded fashion. Mortality caused by fibrosis is shown in this figure.

FIG. 4 is a quantification of liver fibrosis and tissue damage using Image J program and scored using an established clinical ISHAK index from 0 to 4 in a blinded fashion.

FIG. 5. Lung fibrosis and tissue damage were quantified using Image J program and scored using an established clinical index from 0 to 4 in a blinded fashion. Mortality caused by fibrosis is shown in this figure.

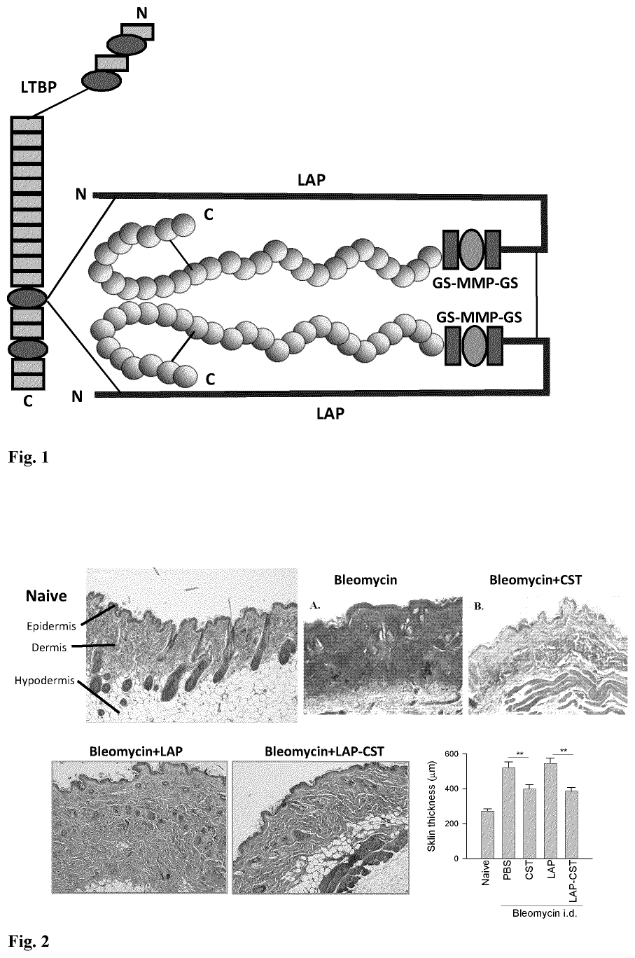

### BRIEF DESCRIPTION OF THE INVENTION

The present invention is directed to a heterologous fusion protein comprising (a) a biologically active protein, fused via (b) a proteolytic cleavage site to (c) a latency associated peptide (LAP) which comprises a precursor domain of TGFβ, wherein said biologically active protein comprises cortistatin or an analogue thereof, wherein said proteolytic cleavage site is a matrix metalloproteinase (MMP) cleavage site and wherein said cortistatin is released from the heterologous fusion protein by MMP-mediated scission. Such fusion protein can be used for any medical need, in particular, for the treatment of chronic fibrosis in a subject in need thereof It is noted that said heterologous fusion protein can be administered to said subject by respiratory, topical, oral, or parenteral administration. It is further noted that such method can be for the treatment of idiopathic fibrosis or any chronic fibrosis selected from the list consisting of liver fibrosis, dermal fibrosis, lung fibrosis, and Scleroderma.

In a preferred embodiment, said matrix metalloproteinase (MMP) cleavage site is cleaved by MMP-9 and flanked by two hydrophilic aminoacidic sequences. In another preferred embodiment, said matrix metalloproteinase (MMP) cleavage site consists of SEQ ID NO 2 and said two hydrophilic aminoacidic sequences, starting from the N-terminus and ending at the C-terminus, are respectively SEQ ID NO 3 and SEQ ID NO 5.

In yet another preferred embodiment, said LAP comprises the precursor domain TGFβ-1, 2, 3, 4 or 5, wherein preferably the latency associated peptide (LAP) consists of SEQ ID NO 1.

In yet another preferred embodiment, said matrix metalloproteinase (MMP) cleavage site consists of SEQ ID NO 2, said two hydrophilic aminoacidic sequences, starting from the N-terminus and ending at the C-terminus, are respectively SEQ ID NO 3 and SEQ ID NO 5 and the latency associated peptide (LAP) consists of SEQ ID NO 1. More preferably, the cortistatin is human cortistatin, preferably of SEQ ID NO 7. Alternatively, the cortistatin is rat cortistatin such as that exemplified by SEQ ID NO 6 or an analogue cortistatin compound of general formula (I),

(I)
R₁-AA₁-AA₂-AA₃-AA₄-c[Cys-AA₅-Asn-X-Y-Trp-Lys-Thr-
Z-AA₆-Ser-Cys]-AA₇-R₂

as well as any stereoisomers, mixtures thereof and/or pharmaceutically acceptable salts, wherein
AA₁ is Asp or a bond
AA₂ is Arg or a bond
AA₃ is Met or Ala or a bond
AA₄ is Pro or Gly
AA₅ is Lys or Arg
AA₆ is Ser or Thr
AA₇ is Lys or a bond
X, Y, Z are the amino acids Phe, Phg, Msa, 3,4,5-trimethylphenylalanine, Msg, 3,4,5-trimethylphenylglycine and/or a dihalogenophenylalanine, diW-Phe;
W is selected from the group consisting of F, Cl, Br and I;
R₁ is selected from the group consisting of H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, a polymer derived from polyethylene glycol, a chelating agent and R₅—CO—;
R₂ is selected from the group consisting of —NR₃R₄, —OR₃ and —SR₃;
R₃ and R₄ are independently selected from the group consisting of H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl and a polymer;
R₅ is selected from the group consisting of H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl;
and with the condition that:
   At least one of the amino acids X, Y or Z is Msa, 3,4,5-trimethylphenylalanine, Msg, 3,4,5-trimethylphenylglycine and/or a dihalogenophenylalanine, diW-Phe;
   If AA₁ and AA₂ are bonds, AA₃ is Ala, AA₄ is Gly, AA₅ is Lys, AA₆ is Thr and AA₇ is a bond, then at least one of the amino acids X, Y or Z is a dihalogenophenylalanine, diW-Phe.

In yet another preferred embodiment, said heterologous fusion protein is of SEQ ID NO 4, or a sequence which has at least 50%, 60%, 70%, 80%, 90%, 95% or 99% identity with a LAP sequence of SEQ ID NO 4, using the default parameters of the BLAST computer program provided by HGMP, thereto.

Further aspects of the present invention refer to a pharmaceutical composition comprising the heterologous fusion protein as defined in any of the above embodiments and, optionally, a pharmaceutically acceptable carrier.

Lastly, it is herein noted that the present invention is not strictly limited to any of the amino acidic sequences mentioned herein, but it can be also reasonably extended to any other sequence having the same function and a structural identity of at least 80%, 85%, 90%, 95% or 99% sequence identity with any of these sequences, using, for example, the default parameters of the BLAST computer program provided by HGMP, thereto.

### DESCRIPTION OF THE INVENTION

According to a first aspect of the invention there is provided a heterologous fusion protein comprising (a) a biologically active protein, fused via (b) a proteolytic cleavage site to (c) a latency associated peptide (LAP) which comprises a precursor domain of TGFβ, wherein said biologically active protein comprises a cortistatin or an analogue thereof. Such fusion protein is capable of significantly increase the blood half-life of cortistatin, as it liberates cortistatin in a controlled-release manner.

The fusion protein comprising a LAP, a proteolytic cleavage site and a pharmaceutically active agent may provide for site specific activation of the latent pharmaceutically active agent. The term “site specific activation” as used herein means, in general terms and not limited to the removal or reduction of latency, conferred on a pharmaceutically active agent, by site-specific cleavage at the proteolytic cleavage site.

Site-specific cleavage at the proteolytic cleavage site is expected to take place concomitantly with the restored activation of the pharmaceutically active agent.

The term “latent pharmaceutically active agent” as used herein refers to a cortistatin or an analogue thereof which are latent due to their association with LAP and a proteolytic cleavage site. Specifically, the cortistatin or an analogue thereof may be latent by virtue of its fusion to a LAP associated proteolytic cleavage site to form a latent fusion protein.

It is noted that an analogue of cortistatin refers to any peptide with anti-inflammatory and/or immunoregulatory action, similar to that of the natural peptide. Certain modifications with non-natural amino acids, such as mesitylalanine and/or dihalogenophenylalanines, plus the incorporation of fatty acids or PEGylations, preserve and even improve the anti-inflammatory and anti-autoimmune action of the natural molecule in vitro and in vivo. In addition, the main benefit of cortistatin analogues is that the synthesis of the cortistatin analogues is economically viable (with sequences of preferably 13 to 17 amino acids), an aspect which guarantees their usefulness in the pharmaceutical industry. In particular, analogues of cortistatins useful in the present invention are defined by formula (I),

(I)
R₁-AA₁-AA₂-AA₃-AA₄-c[Cys-AA₅-Asn-X-Y-Trp-Lys-Thr-
Z-AA₆-Ser-Cys]-AA₇-R₂

its stereoisomers, mixtures thereof and/or its pharmaceutically acceptable salts, wherein
AA₁ is Asp or a bond
AA₂ is Arg or a bond
AA₃ is Met or Ala or a bond
AA₄ is Pro or Gly
AA₅ is Lys or Arg
AA₆ is Ser or Thr
AA₇ is Lys or a bond
X, Y, Z are the amino acids Phe, Phg, Msa, 3,4,5-trimethylphenylalanine, Msg, 3,4,5-trimethylphenylglycine and/or a dihalogenophenylalanine, diW-Phe;
W is selected from the group consisting of F, Cl, Br and I;
R₁ is selected from the group consisting of H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, a polymer derived from polyethylene glycol, a chelating agent and R₅—CO—;
R₂ is selected from the group consisting of —NR₃R₄, —OR₃ and —SR₃;
R₃ and R₄ are independently selected from the group consisting of H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl and a polymer;
R₅ is selected from the group consisting of H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl;
and with the condition that:
   At least one of the amino acids X, Y or Z is Msa, 3,4,5-trimethylphenylalanine, Msg, 3,4,5-trimethylphenylglycine and/or a dihalogenophenylalanine, diW-Phe;
   If AA₁ and AA₂ are bonds, AA₃ is Ala, AA₄ is Gly, AA₅ is Lys, AA₆ is Thr and AA₇ is a bond, then at least one of the amino acids X, Y or Z is a dihalogenophenylalanine, diW-Phe.

In a preferred embodiment, at least one of the amino acids X, Y or Z is a dihalogenophenylalanine, diW-Phe. Preferably, W is fluorine. More preferably the dihalogenophenylalanine is 3,5-difluorophenylalanine (Dfp).

In a preferred embodiment, AA₄ is Pro. In a more preferable embodiment, AA₃ is Met or a bond and AA₄ is Pro. Preferably, at least one of the amino acids X, Y or Z is Msa and/or 3,5-difluorophenylalanine (Dfp).

The R₁ and R₂ groups are bound to the amino-terminal (N-terminal) and carboxy-terminal (C-terminal) ends of the peptide sequences respectively, and they may be amino acids.

In accordance with a preferred embodiment of this invention, R₁ is selected from the group consisting of H, a polymer derived from polyethylene glycol and R₅—CO—, wherein R₅ is selected from the group consisting of substituted or unsubstituted alkyl radical C₁-C₂₄, substituted or unsubstituted alkenyl C₂-C₂₄, substituted or unsubstituted alkynyl C₂-C₂₄, substituted or unsubstituted cycloalkyl C₃-C₂₄, substituted or unsubstituted cycloalkenyl C₅-C₂₄, substituted or unsubstituted cycloalkynyl C₈-C₂₄, substituted or unsubstituted aryl C₆-C₃₀, substituted or unsubstituted aralkyl C₇-C₂₄, substituted or unsubstituted heterocyclyl ring of 3-10 members, and substituted or unsubstituted heteroarylalkyl of 2 to 24 carbon atoms and 1 to 3 atoms other than carbon where the alkyl chain is of 1 to 6 carbon atoms. More preferably, R₁ is selected from the group consisting of H, acetyl, tert-butanoyl, prenyl, hexanoyl, 2-methylhexanoyl, cyclohexanecarboxyl, octanoyl, decanoyl, lauroyl, myristoyl, palmitoyl, stearoyl, behenyl, oleoyl and linoleoyl. Even more preferably, R₁ is H, acetyl, hexanoyl, octanoyl, lauroyl, myristoyl or palmitoyl.

In accordance with another preferred embodiment, R₁ is selected from a polymer derived from polyethylene glycol with a molecular weight comprised between 200 and 35000 Daltons.

In accordance with another preferred embodiment, R₂ is —NR₃R₄, —OR₃ or —SR₃, wherein R₃ and R₄ are independently selected from the group consisting of H, substituted or unsubstituted alkyl C₁-C₂₄, substituted or unsubstituted alkenyl C₂-C₂₄, substituted or unsubstituted alkynyl C₂-C₂₄, substituted or unsubstituted cycloalkyl C₃-C₂₄, substituted or unsubstituted cycloalkenyl C₅-C₂₄, substituted or unsubstituted cycloalkynyl C₈-C₂₄, substituted or unsubstituted aryl C₆-C₃₀, substituted or unsubstituted aralkyl C₇-C₂₄, substituted or unsubstituted heterocyclyl ring of 3-10 members, and substituted or unsubstituted heteroarylalkyl of 2 to 24 carbon atoms and 1 to 3 atoms other than carbon, wherein the alkyl chain is of 1 to 6 carbon atoms and a polymer derived from polyethylene glycol. Optionally, R₃ and R₄ can be bound by a saturated or unsaturated carbon-carbon bond, forming a cycle with the nitrogen atom. More preferably R₂ is —NR₃R₄ or —OR₃, where R₃ and R₄ are independently selected from the group consisting of H, substituted or unsubstituted alkyl C₁-C₂₄, substituted or unsubstituted alkenyl C₂-C₂₄, substituted or unsubstituted alkynyl C₂-C₂₄, substituted or unsubstituted cycloalkyl C₃-C₁₀, substituted or unsubstituted aryl C₆-C₁₅, substituted or unsubstituted heteroarylalkyl ring of 3 to 10 members and an alkyl chain of 1 to 6 carbon atoms and a polymer derived from polyethylene glycol. More preferably R₃ and R₄ are selected from the group consisting of H, methyl, ethyl, hexyl, dodecyl or hexadecyl. Even more preferably R₃ is H and R₄ is selected from the group consisting of H, methyl, ethyl, hexyl, dodecyl or hexadecyl. In accordance with an even more preferred embodiment, R₂ is selected from —OH and —NH₂.

In accordance with a preferred embodiment of this invention, R₁ or R₂ is a chelating agent that is optionally complexed, with a detectable or radio-therapeutic element. A chelating agent refers to a group that is capable of forming coordination complexes with the detectable or radiotherapeutic element. Preferably, the chelating agent is a group capable of forming complexes with metal ions, more preferably selected from the group consisting of DOTA, DTPA, TETA or derivatives thereof. The chelating agent can be bound directly or via a linker.

Detectable element refers to any radioactive, fluorescent or positive contrast magnetic resonance imaging element, preferably a metal ion, which shows a detectable property in an in vivo diagnostic technique. Radiotherapeutic element is understood as any element which emits radiation α, radiation β, or radiation γ.

In a specific embodiment, mora particularly, analogues of cortistatins useful in the present invention are selected from the group of sequences described below (SEQ ID NO 9 to SEQ ID NO 23):

Ala-Gly-c[-Cys-Lys-Asn-Phe-Dfp-Trp-Lys-Thr-Phe-
Thr-Ser-Cys]
Ala-Gly-c[Cys-Lys-Asn-Dfp-Phe-Trp-Lys-Thr-Phe-
Thr-Ser-Cys]
Ala-Gly-c[Cys-Lys-Asn-Phe-Phe-Trp-Lys-Thr-Dfp-
Thr-Ser-Cys]
Ala-Gly-c[Cys-Arg-Asn-Dfp-Phe-Trp-Lys-Thr-Dfp-
Ser-Ser-Cys]
Pro-c[Cys-Lys-Asn-Msa-Phe-Trp-Lys-Thr-Phe-Thr-
Ser-Cys]-Lys
Pro-c[Cys-Lys-Asn-Phe-Msa-Trp-Lys-Thr-Phe-Thr-
Ser-Cys]-Lys
Pro-c[Cys-Lys-Asn-Phe-Dfp-Trp-Lys-Thr-Phe-Thr-
Ser-Cys]-Lys
Pro-c[Cys-Lys-Asn-Phe-Phe-Trp-Lys-Thr-Msa-Thr-
Ser-Cys]-Lys
Pro-c[Cys-Arg-Asn-Msa-Phe-Trp-Lys-Thr-Msa-Thr-
Ser-Cys]-Lys
Pro-c[Cys-Lys-Asn-Dfp-Phe-Trp-Lys-Thr-Msa-Ser-
Ser-Cys]-Lys
Pro-c[Cys-Lys-Asn-Msa-Phe-Trp-Lys-Thr-Phe-Thr-
Ser-Cys]
Pro-c[Cys-Lys-Asn-Phe-Phe-Trp-Lys-Thr-Dfp-Thr-
Ser-Cys]
Met-Pro-c[Cys-Arg-Asn-Msa-Phe-Trp-Lys-Thr-Phe-
Ser-Ser-Cys]-Lys
Asp-Arg-Met-Pro-c[Cys-Arg-Asn-Msa-Phe-Trp-Lys-
Thr-Phe-Thr-Ser-Cys]-Lys
Asp-Arg-Met-Pro-c[Cys-Arg-Asn-Dfp-Phe-Trp-Lys-
Thr-Phe-Thr-Ser-Cys]-Lys

The person skilled in the art will understand that the amino acid sequences referred to in this invention may be chemically modified, for example, by means of chemical modifications that are physiologically relevant, such as phosphorylation, acetylation, amidation, PEGylation, n-octanoylation or palmitoylation, amongst others.

The compounds of this invention can exist as stereoisomers or mixtures of stereoisomers; for example, the amino acids forming them can have a L-, D-configuration, or be racemic independently of one another. Therefore, it is possible to obtain isomeric mixtures, as well as racemic mixtures or diastereomeric mixtures, or pure diastereomers or enantiomers, depending on the number of asymmetric carbons and on which isomers or isomeric mixtures are present. The preferred structures of the peptides of the invention are pure isomers, i.e. a single enantiomer or diastereomer.

For example, unless otherwise indicated, it is understood that the amino acid is L or D, or mixtures thereof, either racemic or non-racemic. The preparation processes described in this document allow the person skilled in the art to obtain each of the stereoisomers of the compound of the invention by choosing the amino acid with the suitable configuration. For example, the amino acid Trp can be L-Trp or D-Trp.

More preferably, the compounds included in formula (I) are selected from the group consisting of:

H-L-Ala-Gly-c[L-Cys-L-Lys-L-Asn-L-Phe-L-Dfp-D-Trp-
L-Lys-L-Thr-L-Phe-L-Thr-L-Ser-L-Cys]-OH
H-L-Ala-Gly-c[L-Cys-L-Lys-L-Asn-L-Dfp-L-Phe-D-Trp-
L-Lys-L-Thr-L-Phe-L-Thr-L-Ser-L-Cys]-OH
H-L-Ala-Gly-c[L-Cys-L-Lys-L-Asn-L-Phe-L-Phe-D-Trp-
L-Lys-L-Thr-L-Dfp-L-Thr-L-Ser-L-Cys]-OH
H-L-Ala-Gly-c[L-Cys-L-Arg-L-Asn-L-Dfp-L-Phe-D-Trp-
L-Lys-L-Thr-L-Dfp-L-Ser-L-Ser-L-Cys]-OH
H-L-Pro-c[L-Cys-L-Lys-L-Asn-L-Msa-L-Phe-D-Trp-L-
Lys-L-Thr-L-Phe-L-Thr-L-Ser-L-Cys]-L-Lys-OH
Octanoyl-L-Pro-c[L-Cys-L-Lys-L-Asn-L-Msa-L-Phe-D-
Trp-L-Lys-L-Thr-L-Phe-L-Thr-L-Ser-L-Cys]-L-Lys-OH
H-L-Pro-c[L-Cys-L-Lys-L-Asn-L-Phe-L-Msa-D-Trp-L-
Lys-L-Thr-L-Phe-L-Thr-L-Ser-L-Cys]-L-Lys-OH
Octanoyl-L-Pro-c[L-Cys-L-Lys-L-Asn-L-Phe-L-Msa-D-
Trp-L-Lys-L-Thr-L-Phe-L-Thr-L-Ser-L-Cys]-L-Lys-OH
Ac-L-Pro-c[L-Cys-L-Lys-L-Asn-L-Phe-L-Dfp-L-Trp-L-
Lys-L-Thr-L-Phe-L-Thr-L-Ser-L-Cys]-L-Lys-NH₂
H-L-Pro-c[L-Cys-L-Lys-L-Asn-L-Phe-L-Phe-D-Trp-L-
Lys-L-Thr-L-Msa-L-Thr-L-Ser-L-Cys]-L-Lys-OH
H-L-Pro-c[L-Cys-L-Arg-L-Asn-L-Msa-L-Phe-D-Trp-L-
Lys-L-Thr-L-Msa-L-Thr-L-Ser-L-Cys]-L-Lys-OH
H-L-Pro-c[L-Cys-L-Lys-L-Asn-L-Dfp-L-Phe-L-Trp-L-
Lys-L-Thr-L-Msa-L-Ser-L-Ser-L-Cys]-L-Lys-NH₂
H-L-Pro-c[L-Cys-L-Lys-L-Asn-L-Msa-L-Phe-D-Trp-L-
Lys-L-Thr-L-Phe-L-Thr-L-Ser-L-Cys]-OH
Octanoyl-L-Pro-c[L-Cys-L-Lys-L-Asn-L-Msa-L-Phe-D-
Trp-L-Lys-L-Thr-L-Phe-L-Thr-L-Ser-L-Cys]-OH
H-L-Pro-c[L-Cys-L-Lys-L-Asn-L-Phe-L-Phe-D-Trp-L-
Lys-L-Thr-L-Dfp-L-Thr-L-Ser-L-Cys]-OH
H-L-Met-L-Pro-c[L-Cys-L-Arg-L-Asn-L-Msa-L-Phe-D-
Trp-L-Lys-L-Thr-L-Phe-L-Ser-L-Ser-L-Cys]-L-Lys-OH
H-L-Asp-L-Arg-L-Met-L-Pro-c[L-Cys-L-Arg-L-Asn-L-
Msa-L-Phe-L-Trp-L-Lys-L-Thr-L-Phe-L-Thr-L-Ser-L-
Cys]-L-Lys-OH
Myristoyl-L-Asp-L-Arg-L-Met-L-Pro-c[L-Cys-L-Arg-
L-Asn-L-Msa-L-Phe-L-Trp-L-Lys-L-Thr-L-Phe-L-Thr-
L-Ser-L-Cys]-L-Lys-OH
H-Asp-L-Arg-L-Met-L-Pro-c[L-Cys-L-Arg-L-Asn-L-
Dfp-L-Phe-D-Trp-L-Lys-L-Thr-L-Phe-L-Thr-L-Ser-
L-Cys]-L-Lys-OH

On the other hand, the term “fusion protein” in this text means, in general terms, one or more proteins joined together by chemical means, including hydrogen bonds or salt bridges, or by peptide bonds through protein synthesis or both.

The latency associated peptide (LAP) of the present invention may include, but is not limited to, the coding sequence for the precursor domain of TGFβ or a sequence which is substantially identical thereto.

“Identity” as known in the art is the relationship between two or more polypeptide sequences or two or more polynucleotide sequences, as determined by comparing the sequences. In the art, identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et al., Nucleic acids Research, 12, 387 (1984), BLASTP, BLASTN, and FASTA (Atschul et al., J. Molec. Biol. 215, 403 (1990).

The LAP of the present invention may comprise the precursor domain of TGFβ, for example, the precursor peptide of TGFβ-1, 2 or 3 (from human) (Derynck et al., Nature, 316, 701-705 (1985); De Martin et al., EMBO J. 6 3673-3677 (1987); Hanks et al., Proc. Natl. Acad. Sci. 85, 79-82 (1988); Derynck et al., EMBO J. 7, 3737-3743 (1988); Ten Dyke et al., Proc. Natl. Acad. Sci. USA, 85, 4715-4719 (1988)) TGFβ-4 (from chicken) (Jakowlew et al., Mol. Endocrinol. 2, 1186-1195 (1988)) or TGFβ-5 (from xenopus) (Kondaiah et al., J. Biol. Chem. 265, 1089-1093 (1990)). The term “precursor domain” is defined as a sequence encoding a precursor peptide which does not include the sequence encoding the mature protein, see sequence SEQ ID NO 1 below

SEQ ID NO 1:
MPPSGLRLLPLLLPLLWLLVLTPGRPAAGLSTCKTIDMELVKRKRIEAI
RGQILSKLRLASPPSQGEVPPGPLPEAVLALYNSTRDRVAGESAEPEPE
PEADYYAKEVTRVLMVETHNEIYDKFKQSTHSIYMFENTSELREAVPEP
VLLSRAELRLLRLKLKVEQHVELYQKYSNNSWRYLSNRLLAPSDSPEWL
SFDVTGVVRQWLSRGGEIEGFRLSAHCSCDSRDNTLQVDINGFTTGRRG
DLATIHGMNRPFLLLMATPLERAQHLQS

At any rate, the amino acid sequences of the precursor domains of TGFβ 1, 2, 3, 4 and 5 are well known (Roberts and Sporn, Peptide Growth Factors and their Receptors: Sporn, M B and Roberts, A B, Springer-Verlag, Chapter 8, 422 (1996)), see also U.S. Pat. No. 8,357,515B2.

Preferably, the amino acid sequence of the LAP has at least 50% identity, using the default parameters of the BLAST computer program (Atschul et al., J. Mol. Biol. 215, 403-410 (1990) provided by HGMP (Human Genome Mapping Project), at the amino acid level, to the precursor domain of TGFβ 1, 2, 3, 4 or 5 (Roberts and Sporn, Peptide Growth Factors and their Receptors: Sporn, M B and Roberts, A B, Springer-Verlag, Chapter 8, 422 (1996)). More preferably, the LAP may have at least 60%, 70%, 80%, 90% and still more preferably 95% (still more preferably at least 99%) identity, at the nucleic acid or amino acid level, to the precursor domain of SEQ ID NO 1 which comprises residues Met1-Ser273.

The LAP may comprise the LAP of TGFβ 1, 2, 3, 4, or 5 (Roberts and Sporn, Peptide Growth Factors and their Receptors: Sporn, M B and Roberts, A B, Springer-Verlag, Chapter 8, 422 (1996)).

The LAP may contain at least two, for example at least 4, 6, 8, 10 or 20 cysteine residues for the formation of disulphide bonds.

The LAP may provide a protective “shell” around the pharmaceutically active agent thereby shielding it and hindering, or preventing, its interaction with other molecules in the cell surface or molecules important for its activity.

The LAP may also comprise a sequence which has at least 50%, 60%, 70%, 80%, 90%, 95% or 99% identity with a LAP sequence of SEQ ID NO 1, using the default parameters of the BLAST computer program provided by HGMP, thereto.

The proteolytic cleavage site may comprise any specific cleavage site which is cleavable by Matrix metalloproteinases (MMPs), also known as matrixins, which are calcium-dependent zinc-containing endopeptidases. In particular, the proteolytic cleavage site is a putative signal peptide for specific cleavage with any of MMP1, MMP2 or MMP9 (PLGLWA), preferably flanked by two hydrophilic aminoacidic sequences (GGGGS (SEQ ID NO 3) and GGGGSAAA (SEQ ID NO 5)) that act as flexible linkers and facilitate entry of the MMP enzyme. More particularly, the proteolytic cleavage site has the following SEQ ID NO 2, or a sequence which has at least 50%, 60%, 70%, 80%, 90%, 95% or 99% identity with sequence of SEQ ID NO 2, using the default parameters of the BLAST computer program provided by HGMP, thereto, and is susceptible of being cleavable by Matrix metalloproteinases (MMPs):

SEQ ID NO 2:
PLGLWA

As already mentioned, the present invention may optionally further provide a “linker” peptide. Preferably the linker peptide is linked to the amino acid sequence of the proteolytic cleavage site. The linker peptide may be provided at the C terminal or N terminal end of the amino acid sequence encoding the proteolytic cleavage site. Preferably, the linker peptide is continuous with the amino acid sequence of the proteolytic cleavage site. The linker peptide may comprise the amino acid sequence GGGGS (SEQ ID NO:3) or a multimer thereof (for example a dimer, a trimer, or a tetramer), a suitable linker may be (GGGGS) (SEQ ID NO:3), or a sequence of aminoacids which has at least 50%, 60%, 70%, 80%, 90%, 95% or 99% identity, using the default parameters of the BLAST computer program provided by HGMP, thereto.

The term “linker peptide” is intended to define any sequence of amino acid residues which preferably provide a hydrophilic region when contained in an expressed protein. Such a hydrophilic region may facilitate cleavage by an enzyme at the proteolytic cleavage site.

The term “latency” as used herein, may relate to a shielding effect which may hinder interaction between the fusion protein and other molecules in the cell surface. Alternatively the term latency may be used to describe a reduction in the activity (up to and including ablation of activity) of a molecule/agent associated with the fusion protein. The term latency may also relate to a stabilising effect of the fusion protein. The effect may be in full or partial, where a partial effect is sufficient to achieve the latency of the active agent.

In a particular preferred embodiment, the fusion protein is SEQ ID NO 4:

SEQ ID NO 4:
MPPSGLRLLPLLLPLLWLLVLTPGRPAAGLSTCKTIDMELVKRKRIEAI
RGQILSKLRLASPPSQGEVPPGPLPEAVLALYNSTRDRVAGESAEPEPE
PEADYYAKEVTRVLMVETHNEIYDKFKQSTHSIYMFFNTSELREAVPEP
VLLSRAELRLLRLKLKVEQHVELYQKYSNNSWRYLSNRLLAPSDSPEWL
SFDVTGVVRQWLSRGGEIEGFRLSAHCSCDSRDNTLQVDINGFTTGRRG
DLATIHGMNRPFLLLMATPLERAQHLQSEFGGGGSPLGLWAGGGGSAAA
QERPPLQQPPHRDKKPCKNFFWKTFSSCK

or a sequence which has at least 50%, 60%, 70%, 80%, 90%, 95% or 99% identity with sequence of SEQ ID NO 4, using the default parameters of the BLAST computer program provided by HGMP, thereto.

The invention further provides nucleic acid encoding the fusion protein of the first aspect of the invention or of any of its preferred embodiments. A second aspect of the invention provides a nucleic acid construct comprising a first nucleic acid sequence encoding for the pharmaceutically active agent, a second nucleic acid sequence encoding a LAP, wherein a nucleic acid sequence encoding a proteolytic cleavage site is provided between the first and second nucleic acid sequences.

The term “nucleic acid construct” generally refers to any length of nucleic acid which may be DNA, cDNA or RNA such as mRNA obtained by cloning or produced by chemical synthesis. The DNA may be single or double stranded. Single stranded DNA may be the coding sense strand, or it may be the non-coding or anti-sense strand. For therapeutic use, the nucleic acid construct is preferably in a form capable of being expressed in the subject to be treated.

The nucleic acid construct of the second aspect of the invention may be in the form of a vector, for example, an expression vector, and may include, among others, chromosomal, episomal and virus-derived vectors, for example, vectors derived from bacterial plasmids, from bacteriophage, from transposons, from yeast episomes, from insertion elements, from yeast chromosomal elements, from viruses such as baculo-viruses, papova-viruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids. Generally, any vector suitable to maintain, propagate or express nucleic acid to express a polypeptide in a host, may be used for expression in this regard.

The invention further provides cortistatin or an analogue thereof encoded by the nucleic acid construct of the second aspect of the invention optionally in association with latent TGFβ binding protein (LTBP) described herein.

The nucleic acid construct of the second aspect of the invention preferably includes a promoter or other regulatory sequence which controls expression of the nucleic acid. Promoters and other regulatory sequences which control expression of a nucleic acid have been identified and are known in the art. The person skilled in the art will note that it may not be necessary to utilise the whole promoter or other regulatory sequence. Only the minimum essential regulatory element may be required and, in fact, such elements can be used to construct chimeric sequences or other promoters. The essential requirement is, of course, to retain the tissue and/or temporal specificity. The promoter may be any suitable known promoter, for example, the human cytomegalovirus (CMV) promoter, the CMV immediate early promoter, the HSV thymidinekinase, the early and late SV40 promoters or the promoters of retroviral LTRs, such as those of the Rous Sarcoma virus (RSV) and metallothionine promoters such as the mouse metallothionine-I promoter. The promoter may comprise the minimum comprised for promoter activity (such as a TATA elements without enhancer elements) for example, the minimum sequence of the CMV promoter.

Preferably, the promoter is contiguous to the first and/or second nucleic acid sequence.

As stated herein, the nucleic acid construct of the second aspect of the invention may be in the form of a vector. Vectors frequently include one or more expression markers which enable selection of cells transfected (or transformed) with them, and preferably, to enable a selection of cells containing vectors incorporating heterologous DNA. A suitable start and stop signal will generally be present.

One embodiment of the invention relates to a cell comprising the nucleic acid construct of the second aspect of the invention. The cell may be termed a “host” cell, which is useful for the manipulation of the nucleic acid, including cloning. Alternatively, the cell may be a cell in which to obtain expression of the nucleic acid. Representative examples of appropriate host cells for expression of the nucleic acid construct of the invention include virus packaging cells which allow encapsulation of the nucleic acid into a viral vector; bacterial cells, such as Streptococci, Staphylococci, E. coli, Streptomyces and Bacillus Subtilis; single cells, such as yeast cells, for example, Saccharomyces Cerevisiae, and Aspergillus cells; insect cells such as Drosophila S2 and Spodoptera Sf9 cells, animal cells such as CHO, COS, C127, 3T3, PHK.293, and Bowes Melanoma cells and other suitable human cells; and plant cells e.g. Arabidopsis thaliana.

Induction of an expression vector into the host cell can be affected by calcium is phosphate transfection, DEAE-dextran mediated transfection, microinjection, cationic-lipid-mediated transfection, electroporation, transduction, scrape loading, ballistic introduction, infection or other methods. Such methods are described in many standard laboratory manuals, such as Sambrook et al, Molecular Cloning, a Laboratory Manual, Second Edition, Coldspring Harbor Laboratory Press, Coldspring Harbor, N.Y. (1989).

Mature proteins can be expressed in host cells, including mammalian cells such as CHO cells, yeast, bacteria, or other cells under the control of appropriate promoters. Cell-free translation systems can be employed to produce such proteins using RNAs derived from the nucleic acid construct of the third aspect of the present invention. Appropriate cloning and expression vectors for use with prokaryotic and eukaryotic hosts are described by Sambrook et al, Molecular Cloning, a Laboratory Manual, Second Edition, Coldspring Harbor Laboratory Press, Coldspring Harbor, N.Y. (1989).

Proteins can be recovered and purified from recombinant cell cultures by well-known methods including ammonium sulphate or ethanol precipitation, acid extraction, anion or cation exchange chromatography, phosphocellulose chromatography, hydrophobic interaction chromatography, affinity chromatography, hydroxylapatite chromatography, high performance liquid chromatography, lectin and/or heparin chromatography. For therapy, the nucleic acid construct e.g. in the form of a recombinant vector, may be purified by techniques known in the art, such as by means of column chromatography as described in Sambrook et al, Molecular Cloning, a Laboratory Manual, Second Edition, Coldspring Harbor Laboratory Press, Coldspring Harbor, N.Y. (1989).

According to a third aspect of the invention, there is provided a composition in accordance with the first aspect of the invention for use in the treatment of chronic fibrosis, preferably selected from the list consisting of liver fibrosis, dermal fibrosis, idiopathic fibrosis, lung fibrosis, and Scleroderma. This aspect of the invention therefore extends to and includes a method for the treatment chronic fibrosis, preferably selected from the list consisting of liver fibrosis, dermal fibrosis, lung fibrosis, and Scleroderma, comprising the administration to a subject of a composition comprising a fusion protein comprising a latency associated peptide (LAP) connected by a proteolytic cleavage site to the pharmaceutically active agent.

In a fourth aspect, the invention provides a nucleic acid sequence in accordance with the second aspect of the invention for use in the treatment of chronic fibrosis, preferably selected from the list consisting of liver fibrosis, dermal fibrosis, lung fibrosis, and Scleroderma. This aspect therefore extends to and includes a method for the treatment of chronic fibrosis, preferably selected from the list consisting of liver fibrosis, dermal fibrosis, lung fibrosis, and Scleroderma, comprising the administration to a subject a nucleic acid construct of the second aspect of the invention. Where the nucleic acid construct is used in the therapeutic method of the invention, the construct may be used as part of an expression construct, e.g. in the form of an expression vector such as a plasmid or virus. In such a method, the construct may be administered intravenously, intradermally, intranasal, intramuscularly, orally or by other routes.

The nucleic acid construct of the second aspect of the invention, and proteins derived therefrom, may be employed alone or in conjunction with other compounds, such as therapeutic compounds, e.g. anti-inflammatory drugs, cytotoxic agents, cytostatic agents or antibiotics. The nucleic acid constructs and proteins useful in the present invention are preferably provided in an isolated form, and preferably are purified to homogeneity.

As used herein, the term “treatment” includes any regime that can benefit a human or a non-human animal. The treatment of “non-human animals” extends to the treatment of domestic animals, including horses and companion animals (e.g. cats and dogs) and farm/agricultural animals including members of the ovine, caprine, porcine, bovine and equine families.

The nucleic acid construct of the second aspect of the invention may be used therapeutically in a method of the invention by way of gene therapy.

Administration of the nucleic acid construct of the second aspect may be directed to the target site by physical methods. Examples of these include topical administration of the “naked” nucleic acid in the form of a vector in an appropriate vehicle, for example, in solution in a pharmaceutically acceptable excipient, such as phosphate buffered saline, or administration of a vector by physical method such as particle bombardment according to methods known in the art.

Other physical methods for administering the nucleic acid construct or proteins of the third aspect of the invention directly to the recipient include ultrasound, electrical stimulation, electroporation and microseeding. Further methods of administration include oral administration or administration through inhalation.

The nucleic acid construct according to the second aspect of the invention may also be administered by means of delivery vectors. These include viral delivery vectors, such as adenovirus, retrovirus or lentivirus delivery vectors known in the art.

Other non-viral delivery vectors include lipid delivery vectors, including liposome delivery vectors known in the art.

Administration may also take place via transformed host cells. Such cells include cells harvested from the subject, into which the nucleic acid construct is transferred by gene transfer methods known in the art. Followed by the growth of the transformed cells in culture and grafting to the subject.

As used herein the term “gene therapy” refers to the introduction of genes by recombinant genetic engineering of body cells (somatic gene therapy) for the benefit of the patient. Furthermore, gene therapy can be divided into ex vivo and in vivo techniques. Ex vivo gene therapy relates to the removal of body cells from a patient, treatment of the removed cells with a vector i.e., a recombinant vector, and subsequent return of the treated cells to the patient. In vivo gene therapy relates to the direct administration of the recombinant gene vector by, for example, intravenous or intravascular means.

Preferably the method of gene therapy of the present invention is carried out ex vivo.

Preferably in gene therapy, the expression vector of the present invention is administered such that it is expressed in the subject to be treated. Thus for human gene therapy, the promoter is preferably a human promoter from a human gene, or from a gene which is typically expressed in humans, such as the promoter from human CMV.

For gene therapy, the present invention may provide a method for manipulating the somatic cells of human and non-human mammals.

The present invention also provides a gene therapy method which may involve the manipulation of the germ line cells of a non-human mammal.

The present invention therefore provides a method for providing a human with a therapeutic protein comprising introducing mammalian cells into a human, the human cells having been treated in vitro to insert therein a nucleic acid construct according to the second aspect of the invention.

Each of the individual steps of the ex vivo somatic gene therapy method are also covered by the present invention. For example, the step of manipulating the cells removed from a patient with the nucleic acid construct of the third aspect of the invention in an appropriate vector. As used herein, the term “manipulated cells” covers cells transfected with a recombinant vector.

Also contemplated is the use of the transfected cells in the manufacture of a medicament for the treatment of chronic fibrosis, preferably selected from the list consisting of liver fibrosis, dermal fibrosis, lung fibrosis, and Scleroderma.

The present invention may also find application in veterinary medicine for treatment/prophylaxis of domestic animals including horses and companion animals (e.g. cats and dogs) and farm animals which may include mammals of the ovine, porcine, caprine, bovine and equine families.

The present invention also relates to compositions comprising the nucleic acid construct or proteins of the first or second aspects of the invention. Therefore, the fusion protein or nucleic acid constructs of the present invention may be employed in combination with the pharmaceutically acceptable carrier or carriers. Such carriers may include, but are not limited to, saline, buffered saline, dextrose, liposomes, water, glycerol, ethanol and combinations thereof.

The pharmaceutical compositions may be administered in any effective, convenient manner effective for treating a patients disease including, for instance, administration by respiratory, oral, topical, intravenous, intramuscular, intranasal, or intradermal routes among others. In therapy or as a prophylactic, the active agent may be administered to an individual as an injectable composition, for example as a sterile aqueous dispersion, preferably isotonic.

For administration to mammals, and particularly humans, it is expected that the daily dosage of the active agent will be from 0.01 mg/kg body weight, typically around 1 mg/kg. The physician in any event will determine the actual dosage which will be most suitable for an individual which will be dependent on factors including the age, weight, sex and response of the individual. The above dosages are exemplary of the average case. There can, of course, be instances where higher or lower dosages are merited, and such are within the scope of this invention

A sixth aspect of the invention provides the fusion protein of the first aspect of the invention, wherein the fusion protein is associated with a pharmaceutically active agent. The pharmaceutically active agent may be as described above.

The present invention also relates to compositions comprising the fusion protein and associated pharmaceutically active agent of the sixth aspect of the invention. Therefore, the fusion protein and associated pharmaceutically active agent may be employed in combination with the pharmaceutically acceptable carrier or carriers. Such carriers may include, but are not limited to, saline, buffered saline, dextrose, liposomes, water, glycerol, polyethylene glycol, ethanol and combinations thereof.

The pharmaceutical compositions may be administered in any effective, convenient manner effective for treating a disease of a patient including, for instance, administration by oral, topical, intravenous, intramuscular, intranasal, or intradermal routes among others. In therapy or as a prophylactic, the active agent may be administered to an individual as an injectable composition, for example as a sterile aqueous dispersion, preferably isotonic.

A seventh aspect of the invention provides for a process for preparing the fusion protein, of the first aspect of the invention comprising production of the fusion protein recombinantly by expression in a host cell, purification of the expressed fusion protein and association of the pharmaceutically active agent to the purified fusion protein by means of peptide bond linkage, hydrogen or salt bond or chemical cross linking.

The following examples are for illustrative purposes only.

### EXAMPLES

Materials and Methods

Characteristics of the Sequences of the Fusion Protein Used in the Examples

SP-LAP-L1-MMP-L2-CST29r (aminoacid sequence)
MPPSGLRLLPLLLPLLWLLVLTPGRPAAGLSTCKTIDMELVKRKRIEAI
RGQILSKLRLASPPSQGEVPPGPLPEAVLALYNSTRDRVAGESAEPEPE
PEADYYAKEVTRVLMVETHNEIYDKFKQSTHSIYMFFNTSELREAVPEP
VLLSRAELRLLRLKLKVEQHVELYQKYSNNSWRYLSNRLLAPSDSPEWL
SFDVTGVVRQWLSRGGEIEGFRLSAHCSCDSRDNTLQVDINGFTTGRRG
DLATIHGMNRPFLLLMATPLERAQHLQSEFGGGGSPLGLWAGGGGSAAA
QERPPLQQPPHRDKKPCKNFFWKTFSSCK*

LAP: latency associated protein of transforming growth factor-B1 (TGFβ-1), Met1-Ser273.

MMP: a putative signal peptide for specific cleavage with MMP1, MMP2 and MMP9 (PLGLWA), flanked by two hydrophilic aminoacidic sequences (GGGGS and GGGGSAAA) that act as flexible linkers and facilitate entry of MMP enzyme. The core of the cleavage site (PLGL) could be substitutes by a different version (PLGI) to be cleaved by MMP3, MMP7 and MMP8.

Cortistatin-29: rat sequence of cortistatin

Other important components:

CSC: Cysteines 224 and 226 are important in the intermolecular disulphide bond between two LAP molecules.

RGD: motif (residues 245-247) facilitates the interaction with integrins.

Cysteine 33: Is important for the disulphide bridge with the third eight-cysteine-rich repeat of latent of latent TGFB binding protein (LTBP).

### Example 1

### Construction of Cortistatin-29 (CST-29) at the C Terminus Followed by GS-MMP-GS and the Fused to Latent Associated Peptide (LAP)

Firstly, we designed a duplex DNA corresponding to the aminoacid sequence from the N-terminal to the C-terminal of LAP, GS-MMP-GS and rat CST-29 (GenBank: EDL81150.1). This duplex DNA was cloned into pCDNA3.1+ (Invitrogen) plasmid digested with EcoRI, usign the In-fusion HD cloning kit (Clontech). This clone was named pCDNA3.1+LAP-GS-MMP-GS-CST-29r (LAP-CST for in vivo experiments) and large amounts of recombinant DNA plasmid was obtained using plasmid Kit endofree columns (Omega). We designed a plasmid containing LAP-GS-MMP-GS, without CST-29r sequence (LAP for in vivo experiments), and was used as control of reference.

Plasmids coding for pCDNA3.1+LAP-GS-MMP-GS-CST-29r or pCDNA3.1+LAP-GS-MMP-GS were transfected into 293T (ATCC Company) cells by using LipoD293™ in vitro transfected reagent (SignaGen Laboratories), and cells were cultured in serum free DMEM during different time periods (24, 48 and 72 h). Culture supernatants were collected and stored at −80° C. until further analysis.

The amount of LAP-MMP-CST-29r peptide in culture supernatants was indirectly determined by measuring the concentration of rat Cortistatin-29 by using a specific ELISA (Phoenix Pharmaceuticals).

293T cells that were transfected with pCDNA3.1+LAP-GS-MMP-GS-CST-29r produced 6.2, 20.3 and 40.2 ng/ml of cortistatin-29 after 24 h, 48 h and 72 h of culture.

293T cells that were transfected with pCDNA3.1+LAP-GS-MMP-GS produced undetectable levels of cortistatin-29 after 24 h, 48 h and 72 h of culture. This suggests that cells transfected with pCDNA3.1+LAP-GS-MMP-GS-CST-29r are able to produce and secrete LAP-CST for a long period of time, so that that we can produce this peptide at high amounts.

Supernatants of 293T cells transfected with pCDNA3.1+LAP-GS-MMP-GS-CST-29r that were collected at 48 h of culture were treated with medium containing recombinant MMP1 and the amount of Cortistatin-29 (indirectly measuring LAP-CST) that was recovered from the culture diminished from 80 ng to 20 ng, 12 hours later; this suggest that cortistatin-29 is cleaved from LAP-CST after exposition to MMP1.

On the other hand, whereas the amount of recombinant cortistatin-29 decreased in a 82% after 15 minutes at room temperature, the amount of cortistatin-29 in LAP-CST remains stable for at least 7 days. This suggests that cortistatin is protected from degradation when is folded in LAP-CST.

### Example 2

### Local Injection of LAP-CST Protects from Dermal Sclerodermia

Sclerodermia was induced by intradermal injection of bleomycin (3 times per week, during four weeks) in an area of 1 cm³ in the dorsal skin of C57Bl/6 mice. Mice were locally treated around the lesion area with saline (group bleomycin), with cortistatin (group belomycin+CST, 3 time per week, 10 ng each time), with empty LAP vector (Bleomycin+LAP, once a week, 20 pg) or with LAP-CST (Bleomycin+LAP-CST, once a week, 20 pg). Naïve animals without bleomycin were used as basal control reference. After four weeks, lesioned skin area was dissected and processed for histological analysis using Mason Trichromic staining. Skin thickness (from epidermis to hypodermis) was quantified using Image J program. Fibrotic deposits in skin are stained in blue in sections.

These results indicate that treatment with LAP-CST is effective in reducing dermal fibrosis induced by bleomycin, in comparison to treatment with LAP alone. Moreover, weekly treatment with LAP-CST was as effective as the repetitive treatment (3 times per week) with recombinant cortistatin used at 500-fold higher concentration, suggesting that a single injection of LAP-CST is at least 1,500 times more effective than recombinant cortistatin.

### Example 3

### Treatment with LAP-CST Protects from Lung Fibrosis

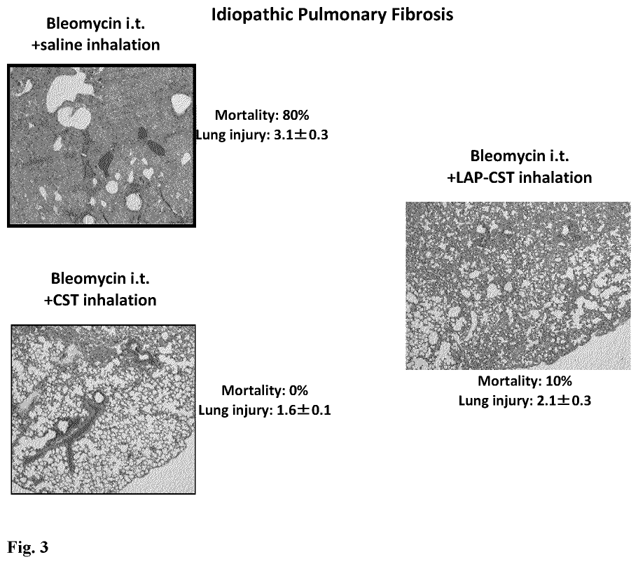

Lung fibrosis was induced by intratracheal injection of bleomycin (50 μg/kg body weight, dissolved in 50 μl of saline) in C57Bl/6 mice. Mice were treated by nasal inhalation of saline (group bleomycin+saline), cortistatin (group belomycin+CST, 3 times per week, 10 ng each time), or LAP-CST (Bleomycin+LAP-CST, once a week, 20 pg). After 18 days, lungs were dissected and processed for histological analysis using Mason Trichromic staining and Sirius Red staining. Lung fibrosis and tissue damage were quantified using Image J program and scored using an established clinical index from 0 to 4 in a blinded fashion. Mortality caused by fibrosis is shown in FIG. 3.

These results indicated that intratracheal injection of bleomycin induces severe idiopathic pulmonary fibrosis in mice that causes 80% of mortality. Treatment with LAP-CST by nasal inhalation significantly reduced lung fibrosis and damage as well as dramatically increased survival. LAP-CST treatment was as effective as repetitive inhalation of recombinant cortistatin peptide (3 times per week, at a 500-fold higher dose).

### Example 4

### Treatment with LAP-CST Protects from Liver Fibrosis

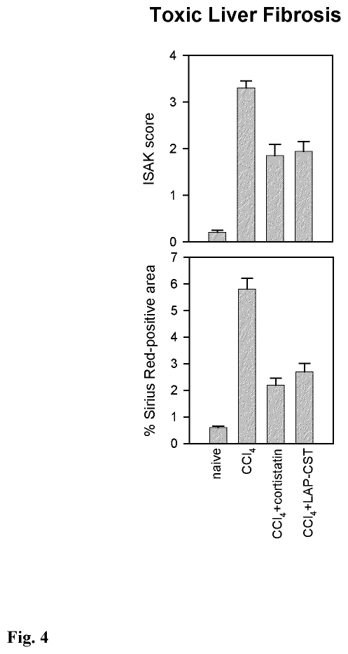

Liver fibrosis was induced by intraperitoneal injection of CCl4 (3:9 in olive oil, 40 μl/mouse, every three days) in C57Bl/6 mice. Mice were treated three times a week with cortistatin (500 ng/mouse) or once a week with LAP-CST (100 pg/mouse). After 6 weeks, liver was dissected and processed for histological analysis using Mason Trichromic staining and Sirius Red staining. Liver fibrosis and tissue damage were quantified using Image J program and scored using an established clinical ISHAK index from 0 to 4 in a blinded fashion (FIG. 4).

Chronic injection of CCl4 induced an extensive parenchymal fibrosis in the liver that caused a mortality of 50% in untreated mice. Systemic treatment with LAP-CST significantly reduced liver fibrotic area and avoided mortality (100% survival) in a similar way than cortistatin.

### Example 5

### Treatment with LAP-CST Protects from Lung Fibrosis

Lung fibrosis was induced by intratracheal injection of bleomycin (50 μg/kg body weight, dissolved in 50 μl of saline) in C57Bl/6 mice. Mice were treated once a week by nasal inhalation of 20 pg of empty LAP (A), LAP containing only cortistatin (B), LAP containing cortistatin and linkers LAP-L1L2-CST but not MMP site (C), or LAP containing cortistatin, linkers and MMP site (D). After 18 days, lungs were dissected and processed for histological analysis using Mason Trichromic staining and Sirius Red staining. Lung fibrosis and tissue damage were quantified using Image J program and scored using an established clinical index from 0 to 4 in a blinded fashion. Mortality caused by fibrosis is shown in FIG. 5.

Only the injection of LAP containing cortistatin, linkers and MMP site protected versus bleomycin-induced lung fibrosis and mortality. These results indicated that cortistatin needs to be released from LAP by MMP-mediated scission in the fibrotic tissue to be therapeutically effective in chronic fibrosis.

1. A heterologous fusion protein comprising (a) a biologically active protein, fused via (b) a proteolytic cleavage site to (c) a latency associated peptide (LAP) which comprises a precursor domain of TGFβ, wherein said biologically active protein is cortistatin or an analogue thereof, wherein said proteolytic cleavage site is a matrix metalloproteinase (MMP) cleavage site and wherein said cortistatin is released from the heterologous fusion protein by MMP-mediated scission, for use in the treatment of chronic fibrosis.

2. The heterologous fusion protein for use according to claim 1, wherein said matrix metalloproteinase (MMP) cleavage site is cleaved by MMP-9 and flanked by two hydrophilic aminoacidic sequences.

3. The heterologous fusion protein for use according to claim 2, wherein said matrix metalloproteinase (MMP) cleavage site consists of SEQ ID NO 2 and wherein said two hydrophilic aminoacidic sequences, starting from the N-terminus and ending at the C-terminus, are respectively SEQ ID NO 3 and SEQ ID NO 5.

4. The heterologous fusion protein for use according to any of claims 1 to 3, wherein said LAP comprises the precursor domain TGFμ-1, 2, 3, 4 or 5.

5. The heterologous fusion protein for use according to claim 4, wherein the latency associated peptide (LAP) consists of SEQ ID NO 1.

6. The heterologous fusion protein for use according to any of claims 1 to 5, wherein said matrix metalloproteinase (MMP) cleavage site consists of SEQ ID NO 2, wherein said two hydrophilic aminoacidic sequences, starting from the N-terminus and ending at the C-terminus, are respectively SEQ ID NO 3 and SEQ ID NO 5 and wherein the latency associated peptide (LAP) consists of SEQ ID NO 1.

7. The heterologous fusion protein for use according to any of claims 1 to 6, wherein the cortistatin is human cortistatin, preferably of SEQ ID NO 7.

8. The heterologous fusion protein for use according to claim 6, wherein the cortistatin is human cortistatin, preferably of SEQ ID NO 7.

9. The heterologous fusion protein for use according to any of claims 1 to 6, wherein the cortistatin consists of SEQ ID NO 6.

10. The heterologous fusion protein for use according to any of claims 1 to 6, wherein the analogue cortistatin compound is of general formula (I),
(I)
R₁-AA₁-AA₂-AA₃-AA₄-c[Cys-AA₅-Asn-X-Y-Trp-Lys-Thr-
Z-AA₆-Ser-Cys]-AA₇-R₂
wherein
AA₁ is Asp or a bond
AA₂ is Arg or a bond
AA₃ is Met or Ala or a bond
AA₄ is Pro or Gly
AA₅ is Lys or Arg
AA₆ is Ser or Thr
AA₇ is Lys or a bond
X, Y, Z are the amino acids Phe, Phg, Msa, 3,4,5-trimethylphenylalanine, Msg, 3,4,5-trimethylphenylglycine and/or a dihalogenophenylalanine, diW-Phe;
W is selected from the group consisting of F, Cl, Br and I;
R₁ is selected from the group consisting of H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, substituted or unsubstituted aralkyl, a polymer derived from polyethylene glycol, a chelating agent and R₅—CO—;
R₂ is selected from the group consisting of —NR₃R₄, —OR₃ and —SR₃;
R₃ and R₄ are independently selected from the group consisting of H, a non-cyclic substituted or unsubstituted aliphatic group, substituted or unsubstituted alicyclyl, substituted or unsubstituted heterocyclyl, substituted or unsubstituted heteroarylalkyl, substituted or unsubstituted aryl, and substituted or unsubstituted aralkyl and a polymer;
R₅ is selected from the group consisting of H, a non-cyclic substituted or unsubstituted aliphatic group, substituted or unsubstituted alicyclyl, substituted or unsubstituted aryl, substituted or unsubstituted aralkyl, substituted or unsubstituted heterocyclyl and substituted or unsubstituted heteroarylalkyl;
and with the condition that:
At least one of the amino acids X, Y or Z is Msa, 3,4,5-trimethylphenylalanine, Msg, 3,4,5-trimethylphenylglycine and/or a dihalogenophenylalanine, diW-Phe;
If AA₁ and AA₂ are bonds, AA₃ is Ala, AA₄ is Gly, AA₅ is Lys, AA₆ is Thr and AA₇ is a bond, then at least one of the amino acids X, Y or Z is a dihalogenophenylalanine, diW-Phe.

11. The heterologous fusion protein for use according to claim 1, wherein said fusion protein is SEQ ID NO 4, or a sequence which has at least 95% sequence identity with a LAP sequence of SEQ ID NO 4, using the default parameters of the BLAST computer program provided by HGMP, thereto.

12. A pharmaceutical composition comprising the heterologous fusion protein as defined in any of claims 1 to 11 and a pharmaceutically acceptable carrier.

13. The heterologous fusion protein for use according to any of claims 1 to 11, wherein said heterologous fusion protein is administered to said mammal by respiratory, topical, oral, or parenteral administration.

14. The heterologous fusion protein for use according to any of claim 1 to 11 or 13, wherein the method is for the treatment of idiopathic fibrosis.

15. The heterologous fusion protein for use according to any of claim 1 to 11 or 13, wherein said chronic fibrosis is selected from the list consisting of liver fibrosis, dermal fibrosis, lung fibrosis, and Scleroderma.
